(19) United States
(12) Patent Application Publication
(10) Pub. No.: US 2025/0180464 A1
(43) Pub. Date: Jun. 5, 2025

(54) METHOD FOR SORTING PHOTOSYNTHETIC DYE-CONTAINING CELL, CELL SORTER, AND CELL SORTING SYSTEM

(71) Applicant: Sony Group Corporation, Tokyo (JP)

(72) Inventors: Ayaka Kuroyama, Kanagawa (JP); Yoshiharu Hayashi, Kanagawa (JP); Sousuke Imamura, Tokyo (JP); Nozomi Takeue, Tokyo (JP); Kan Tanaka, Tokyo (JP)

(73) Assignee: Sony Group Corporation, Tokyo (JP)

(21) Appl. No.: 18/717,611

(22) PCT Filed: Nov. 29, 2022
(86) PCT No.: PCT/JP2022/043930

§ 371 Date: Dec. 17, 2024

(30) Foreign Application Priority Data
Dec. 16, 2021 (JP) 2021-204087

(51) Int. Cl. G01N 15/149; G01N 21/64

(52) U.S. Cl. CPC G01N 15/149; G01N 21/64; G01N 2333/405; G01N 2333/415

(57) ABSTRACT

An object of the present disclosure is to provide a technique for more efficiently obtaining a photosynthetic dye-containing cell having a desired characteristic.

The present disclosure is directed to providing a method for sorting a photosynthetic dye-containing cell, including a measurement step of measuring autofluorescence spectra of the cultured photosynthetic dye-containing cell group; and a sorting step of sorting a cell population having a suppressed photosynthetic-dye decrease from the photosynthetic dye-containing cell group on the basis of the measured autofluorescence spectra. The present disclosure is also directed to providing a cell sorter and a cell sorting system configured to perform the sorting method.

### TECHNICAL FIELD

The present disclosure relates to a method for sorting photosynthetic dye-containing cells, a device for sorting the cells, and a system for sorting the cells. More specifically, the present disclosure relates to a method for sorting photosynthetic dye-containing cells to be performed for sorting a cell population having a suppressed photosynthetic-dye decrease, and a cell sorter and a cell sorting system configured to perform the sorting method.

### BACKGROUND ART

Techniques for reproducibly producing fuels or chemical products by photosynthesis using photosynthetic dye-containing cells such as algal cells and plant cells have attracted attention. For this technique, studies have been conducted to enhance the solar energy conversion efficiency of cells containing a photosynthetic dye. For example, Non Patent Document 1 shown below discloses that deletion of a specific gene in a specific green algae can be employed for producing a variant having a reduced chlorophyll antenna size.

### CITATION LIST

### Non Patent Document

Non Patent Document 1: Kirst et al., Plant Physiology, Volume 160, Issue 4, December 2012, Pages 2251-2260

### SUMMARY OF THE INVENTION

### Problems to be Solved by the Invention

As described above, it has been studied to use photosynthetic dye-containing cells such as algal cells and plant cells for photosynthetically producing useful chemicals. Examples of the useful chemicals include lipids and/or carbohydrates produced by photosynthesis. These are useful biomass and can be used for producing, for example, biofuels.

For producing such useful chemicals, the photosynthetic dye-containing cells are cultured under specific conditions. However, in such culture, a decrease of a photosynthetic dye often causes a problem. The decrease of the photosynthetic dye sometimes occurs, for example, in a case of culturing cells under a nitrogen-deficient environment, and undesirable for producing a desired substance. Therefore, it is considered that selectively obtaining cells having a suppressed photosynthetic-dye decrease can contribute to efficient production of useful chemical substances.

For selectively obtaining cells as described above, culturing a cell under specific conditions and obtaining a desired cell is performed. In such culture, a cell is often cultured on a predetermined medium. However, in order to obtain a desired cell, it is necessary to perform culture using a large number of plates containing a culture medium, and further such culture is often repeated many times. Therefore, the conventional process for obtaining a desired cell is complicated and often takes time.

An object of the present disclosure is to provide a technique for more efficiently obtaining a photosynthetic dye-containing cell having a desired characteristic.

### Solutions to Problems

The present disclosure is directed to providing
a method for sorting a photosynthetic dye-containing cell, including
a measurement step of measuring autofluorescence spectra of the cultured photosynthetic dye-containing cell group; and
a sorting step of sorting a cell population having a suppressed photosynthetic-dye decrease from the photosynthetic dye-containing cell group on the basis of the measured autofluorescence spectra.

In the measurement step, the autofluorescence spectra may be measured for at least two photosynthetic dye-containing cell group samples different in characteristic.

The difference in characteristic may be ascribed to a difference in culture period.

The cell population having a suppressed photosynthetic-dye decrease may be a cell population identified on the basis of a difference in autofluorescence spectra of the at least two photosynthetic dye-containing cell group samples.

The cell population having a suppressed photosynthetic-dye decrease may be identified by setting a gate on data of the measured autofluorescence spectra.

The culture may be culture to be performed in a culture environment in which a photosynthetic dye within a cell is induced to decrease.

The culture environment may be a culture environment in which a medium containing components adjusted so as to induce a decrease of a photosynthetic dye within a cell is used, or a culture environment in which irradiation light to cells is adjusted so as to induce a decrease of a photosynthetic dye within a cell.

The sorting method may further include an identification step of identifying a cell population having a suppressed photosynthetic-dye decrease on the basis of the measured autofluorescence spectra, and
the cell population identified in the identification step may be sorted in the sorting step. The identification step may be performed by a cell analyzer, and the sorting step may be performed by a cell sorter, or
both the identification step and the sorting step may be performed by a cell sorter.

The photosynthetic dye-containing cell may be an algal cell or a plant cell.

The photosynthetic dye-containing cell may be a microalgal cell or a plant cell.

The photosynthetic dye-containing cell may be an oxygenic photosynthetic cell or may be a non-oxygenic photosynthetic cell.

The photosynthetic dye may be a dye possessed by a photosynthetic organism.

The photosynthetic dye may contain at least one of a phycobilin dye, a chlorophyll dye, and a carotenoid dye.

Furthermore, the present disclosure is directed to providing
a method for sorting a photosynthetic dye-containing cell, including
a sorting step of sorting a cell population having a suppressed photosynthetic-dye decrease from a photosynthetic dye-containing cell group, in which
the cell population having a suppressed photosynthetic-dye decrease is a cell population identified on the basis of autofluorescence spectra of the cultured photosynthetic dye-containing cell group.

Furthermore, the present disclosure is directed to providing
a cell sorter configured to
perform a sorting step of sorting a cell population having a suppressed photosynthetic-dye decrease from the photosynthetic dye-containing cell group, in which
the cell population having a suppressed photosynthetic-dye decrease is a cell population identified on the basis of the autofluorescence spectra of the cultured photosynthetic dye-containing cell group.

Furthermore, the present disclosure is directed to providing
a cell sorting system including
a cell sorter configured to perform a sorting step of sorting a cell population having a suppressed photosynthetic-dye decrease from the photosynthetic dye-containing cell group, in which
the cell population having a suppressed photosynthetic-dye decrease is a cell population identified on the basis of the autofluorescence spectra of the cultured photosynthetic dye-containing cell group.

### BRIEF DESCRIPTION OF DRAWINGS

FIG. 1 is a diagram schematically showing the overall configuration of a biological sample analyzer.

FIG. 2 is a flowchart of the sorting method according to the present disclosure.

FIG. 3 illustrates a photograph of a culture solution obtained by culturing for 0 week, 1 week, 2 weeks, or 4 weeks.

FIG. 4A illustrates views showing measurement results of autofluorescence spectra.

FIG. 4B is a view showing measurement results of autofluorescence spectra.

FIG. 5A is a diagram for illustrating identification of a cell population having a suppressed photosynthetic-dye decrease.

FIG. 5B is a view showing measurement results of average autofluorescence spectra.

FIG. 6 illustrates measurement results of autofluorescence spectra of −N week 0 and −N week 4 by a cell sorter.

FIG. 7A illustrates graphs showing measurement results of autofluorescence spectra of sorted cell populations by a spectral analyzer.

FIG. 7B illustrates a graph showing measurement results of average autofluorescence spectra.

FIG. 8 illustrates a block diagram of the cell sorter according to the present disclosure.

FIG. 9A illustrates a block diagram of the cell sorting system according to the present disclosure.

FIG. 9B is a block diagram of the cell sorter contained in the cell sorter system according to the present disclosure.

FIG. 9C is a block diagram of the cell sorter contained in the cell sorter system according to the present disclosure.

### MODE FOR CARRYING OUT THE INVENTION

Hereinafter, preferred modes for carrying out the present disclosure will be described. Note that embodiments described below illustrate representative embodiments of the present disclosure, and the scope of the present disclosure is not limited to these embodiments alone. Note that the present disclosure will be described in the following order.

### 1. Method for Sorting Photosynthetic Dye-Containing Cell

(1) Outline of sorting method according to the present disclosure
(2) Examples of configuration of device for performing the sorting method according to the present disclosure
(3) Example of sorting method according to the present disclosure
(3-1) Measurement step
(3-1-1) Culture
(3-1-2) Measurement of autofluorescence spectra
(3-1-3) Examples of samples to be measured
(3-1-4) Examples of cells
(2-3) Identification step
(3-2-1) Examples of identifying desired cell population
(3-2-2) Examples of identification of fluorescence wavelength band for identifying desired cell population
(3-2-3) Examples of gate setting for identifying desired cell population
(3-3) Sorting step
(4) Examples
2. Cell Sorter
3. Cell Sorting System

### 1. Method for Sorting Photosynthetic Dye-Containing Cell

### (1) Outline of Sorting Method According to the Present Disclosure

The present inventors have found that the autofluorescence spectra of a photosynthetic dye-containing cell can be used for sorting a cell having a desired characteristic, more particularly, for sorting a cell population having a suppressed photosynthetic-dye decrease. That is, the present disclosure provides a cell sorting method for sorting a cell population having a desired characteristic (particularly, a cell population having a suppressed photosynthetic-dye decrease) from a photosynthetic dye-containing cell group on the basis of the autofluorescence spectra of photosynthetic dye-containing cells.

In an embodiment, the sorting method according to the present disclosure includes a measurement step of measuring autofluorescence spectra of a cultured photosynthetic dye-containing cell group; and a sorting step of sorting a cell population having a suppressed photosynthetic-dye decrease from the photosynthetic dye-containing cell group on the basis of the measured autofluorescence spectra. A cell population having a desired characteristic can be selectively obtained by performing these steps.

For example, in the measurement step, autofluorescence spectra of samples of at least two photosynthetic dye-containing cell group different in characteristic may be measured. In this manner, it is possible to identify a difference in the autofluorescence spectrum between a cell population having, for example, a desired characteristic, and a cell population not having the desired characteristic.

The difference in characteristic may be ascribed to, e.g., a difference in culture period. For example, in a case where the culture period under the nitrogen-deficient condition is 0 day (that is, in a case where the culture does not proceed under the nitrogen-deficient condition), the photosynthetic dye in algal cells or plant cells does not decrease, but the photosynthetic dye may sometimes decrease by performing culture under the condition for several weeks. Then, in a case where there is a cell population having no decrease of a photosynthetic dye among the cell populations cultured under the condition for several weeks, the cell population having no decrease of a photosynthetic dye can be identified as a cell population having a suppressed photosynthetic-dye decrease. The identification can be performed on the basis of the measured autofluorescence spectra. Such a cell population is a cell population having a suppressed photosynthetic-dye decrease in culture under a nitrogen-deficient condition, and is useful, in a case where, for example, a nitrogen-deficient condition is employed for production of a desired substance.

### (2) Examples of Configuration of Device for Performing the Sorting Method According to the Present Disclosure

In one embodiment, the sorting method according to the present disclosure may be performed using a cell analyzer configured to enable measurement of autofluorescence spectra, and a cell sorter for sorting cells to be sorted identified on the basis of the measured autofluorescence spectra. In this embodiment, a spectral analyzer may be used as the former device and a cell sorter may be used as the latter device.

In another embodiment, the sorting method according to the present disclosure is configured to be capable of measuring autofluorescence spectra, and may be performed using a cell sorter that performs sorting of the cells to be sorted identified on the basis of the measurement results. In this embodiment, a spectral cell sorter may be used.

These cell analyzer and cell sorter may be configured as a biological sample analyzer that will be described below. That is, these devices may contain components (flow path, light irradiation unit, detection unit, and information processing unit, and optionally, sorting unit) contained in the biological sample analyzer that will be described below.

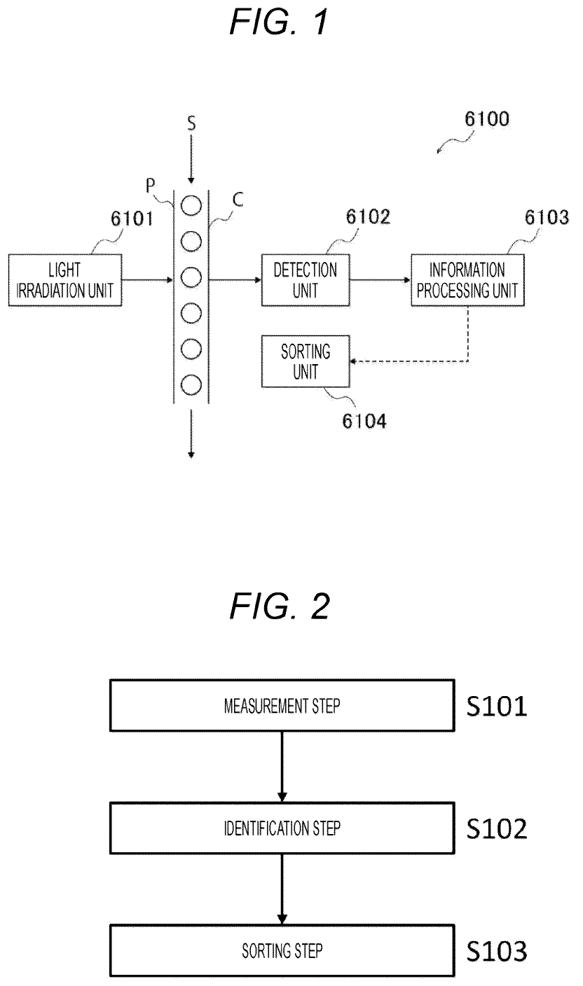

A configuration of a biological sample analyzer of the present disclosure is shown in FIG. 1. A biological sample analyzer 6100 shown in FIG. 1 includes: a light irradiation unit 6101 configured to apply light to a biological sample S flowing in a flow channel C; a detection unit 6102 configured to detect light generated by applying light to the biological sample S; and an information processing unit 6103 configured to process information about the light detected by the detection unit. The biological sample analyzer 6100 is a flow cytometer or an imaging cytometer, for example. The biological sample analyzer 6100 may include a sorting unit 6104 that sorts out specific biological particles P in a biological sample. The biological sample analyzer 6100 including the sorting unit is a cell sorter, for example.

### (Biological Sample)

The biological sample S may be a liquid sample containing biological particles. The biological particles are cells or non-cellular biological particles, for example. The cells may be living cells, and more specific examples thereof include blood cells such as erythrocytes and leukocytes, and germ cells such as sperms and fertilized eggs. Also, the cells may be those directly collected from a sample such as whole blood, or may be cultured cells obtained after culturing. The non-cellular biological particles are extracellular vesicles, or particularly, exosomes and microvesicles, for example. The biological particles may be labeled with one or more labeling substances (such as a dye (particularly, a fluorescent dye) and a fluorochrome-labeled antibody). Note that particles other than biological particles may be analyzed by the biological sample analyzer of the present disclosure, and beads or the like may be analyzed for calibration or the like.

### (Flow Channel)

The flow channel C is designed so that a flow of the biological sample S is formed. In particular, the flow channel C may be designed so that a flow in which the biological particles contained in the biological sample are aligned substantially in one row is formed. The flow channel structure including the flow channel C may be designed so that a laminar flow is formed. In particular, the flow channel structure is designed so that a laminar flow in which the flow of the biological sample (a sample flow) is surrounded by the flow of a sheath liquid is formed. The design of the flow channel structure may be appropriately selected by a person skilled in the art, or a known one may be adopted. The flow channel C may be formed in a flow channel structure such as a microchip (a chip having a flow channel on the order of micrometers) or a flow cell. The width of the flow channel C is 1 mm or smaller, or particularly, may be not smaller than 10 μm and not greater than 1 mm. The flow channel C and the flow channel structure including the flow channel C may be made of a material such as plastic or glass.

The biological sample analyzer of the present disclosure is designed so that the biological sample flowing in the flow channel C, or particularly, the biological particles in the biological sample are irradiated with light from the light irradiation unit 6101. The biological sample analyzer of the present disclosure may be designed so that the irradiation point of light on the biological sample is located in the flow channel structure in which the flow channel C is formed, or may be designed so that the irradiation point is located outside the flow channel structure. An example of the former case may be a configuration in which the light is emitted onto the flow channel C in a microchip or a flow cell. In the latter case, the biological particles after exiting the flow channel structure (particularly, the nozzle portion thereof) may be irradiated with the light, and a flow cytometer of a jet-in-air type can be adopted, for example.

### (Light Irradiation Unit)

The light irradiation unit 6101 includes a light source unit that emits light, and a light guide optical system that guides the light to the irradiation point. The light source unit includes one or more light sources. The type of the light source(s) is a laser light source or an LED, for example. The wavelength of light to be emitted from each light source may be any wavelength of ultraviolet light, visible light, and infrared light. The light guide optical system includes optical components such as beam splitters, mirrors, or optical fibers, for example. The light guide optical system may also include a lens group for condensing light, and includes an objective lens, for example. There may be one or more irradiation points at which the biological sample and light intersect. The light irradiation unit 6101 may be designed to collect light emitted onto one irradiation point from one light source or different light sources.

### (Detection Unit)

The detection unit 6102 includes at least one photodetector that detects light generated by emitting light onto biological particles. The light to be detected may be fluorescence or scattered light (such as one or more of the following: forward scattered light, backscattered light, and side scattered light), for example. Each photodetector includes one or more light receiving elements, and has a light receiving element array, for example. Each photodetector may include one or more photomultiplier tubes (PMTs) and/or photodiodes such as APDs and MPPCs, as the light receiving elements. The photodetector includes a PMT array in which a plurality of PMTs is arranged in a one-dimensional direction, for example. The detection unit 6102 may also include an image sensor such as a CCD or a CMOS. With the image sensor, the detection unit 6102 can acquire an image (such as a bright-field image, a dark-field image, or a fluorescent image, for example) of biological particles.

The detection unit 6102 includes a detection optical system that causes light of a predetermined detection wavelength to reach the corresponding photodetector. The detection optical system includes a spectroscopic unit such as a prism or a diffraction grating, or a wavelength separation unit such as a dichroic mirror or an optical filter. The detection optical system is designed to disperse the light generated by light irradiation to biological particles, for example, and detect the dispersed light with a larger number of photodetectors than the number of fluorescent dyes with which the biological particles are labeled. A flow cytometer including such a detection optical system is called a spectral flow cytometer. Further, the detection optical system is designed to separate the light corresponding to the fluorescence wavelength band of a specific fluorescent dye from the light generated by the light irradiation to the biological particles, for example, and cause the corresponding photodetector to detect the separated light.

The detection unit 6102 may also include a signal processing unit that converts an electrical signal obtained by a photodetector into a digital signal. The signal processing unit may include an A/D converter as a device that performs the conversion. The digital signal obtained by the conversion performed by the signal processing unit can be transmitted to the information processing unit 6103. The digital signal can be handled as data related to light (hereinafter, also referred to as “light data”) by the information processing unit 6103. The light data may be light data including fluorescence data, for example. More specifically, the light data may be data of light intensity, and the light intensity may be light intensity data of light including fluorescence (the light intensity data may include feature quantities such as area, height, and width).

### (Information Processing Unit)

The information processing unit 6103 includes a processing unit that performs processing of various kinds of data (light data, for example), and a storage unit that stores various kinds of data, for example. In a case where the processing unit acquires the light data corresponding to a fluorescent dye from the detection unit 6102, the processing unit can perform fluorescence leakage correction (a compensation process) on the light intensity data. In the case of a spectral flow cytometer, the processing unit also performs a fluorescence separation process on the light data, and acquires the light intensity data corresponding to the fluorescent dye. The fluorescence separation process may be performed by an unmixing method disclosed in JP 2011-232259 A, for example. In a case where the detection unit 6102 includes an image sensor, the processing unit may acquire morphological information about the biological particles, on the basis of an image acquired by the image sensor. The storage unit may be designed to be capable of storing the acquired light data. The storage unit may be designed to be capable of further storing spectral reference data to be used in the unmixing process.

In a case where the biological sample analyzer 6100 includes the sorting unit 6104 described later, the information processing unit 6103 can determine whether to sort the biological particles, on the basis of the light data and/or the morphological information. The information processing unit 6103 then controls the sorting unit 6104 on the basis of the result of the determination, and the biological particles can be sorted by the sorting unit 6104.

The information processing unit 6103 may be designed to be capable of outputting various kinds of data (such as light data and images, for example). For example, the information processing unit 6103 can output various kinds of data (such as a two-dimensional plot or a spectrum plot, for example) generated on the basis of the light data. The information processing unit 6103 may also be designed to be capable of accepting inputs of various kinds of data, and accepts a gating process on a plot by a user, for example. The information processing unit 6103 may include an output unit (such as a display, for example) or an input unit (such as a keyboard, for example) for performing the output or the input.

The information processing unit 6103 may be designed as a general-purpose computer, and may be designed as an information processing device that includes a CPU, a RAM, and a ROM, for example. The information processing unit 6103 may be included in the housing in which the light irradiation unit 6101 and the detection unit 6102 are included, or may be located outside the housing. Further, the various processes or functions to be executed by the information processing unit 6103 may be realized by a server computer or a cloud connected via a network.

### (Sorting Unit)

The sorting unit 6104 performs sorting of biological particles, in accordance with the result of determination performed by the information processing unit 6103. The sorting method may be a method by which droplets containing biological particles are generated by vibration, electric charges are applied to the droplets to be sorted, and the traveling direction of the droplets is controlled by an electrode. The sorting method may be a method for sorting by controlling the traveling direction of biological particles in the flow channel structure. The flow channel structure has a control mechanism based on pressure (injection or suction) or electric charge, for example. An example of the flow channel structure may be a chip (the chip disclosed in JP 2020-76736 A, for example) that has a flow channel structure in which the flow channel C branches into a recovery flow channel and a waste liquid flow channel on the downstream side, and specific biological particles are collected in the recovery flow channel.

### (3) Example of Sorting Method According to the Present Disclosure

A flowchart of a sorting method according to the present disclosure is shown in FIG. 2. As shown in the figure, the sorting method includes a measurement step S1, an identification step S2, and a sorting step S3. These steps may be performed by the biological sample analyzer described above.

In one embodiment, the sorting method may be carried out by, for example, a single biological sample analyzer. In this embodiment, the biological sample analyzer used in the sorting method may be a cell sorter configured to be capable of measuring autofluorescence spectra of cells. Examples of the cell sorter include a spectral cell sorter. In this embodiment, all of the measurement step S1, the identification step S2, and the sorting step S3 may be performed using the cell sorter.

In another embodiment, the sorting method may be carried out by, for example, two or more biological sample analyzers (particularly two biological sample analyzers). In this embodiment, the two or more biological sample analyzers to be used in the sorting method includes a cell analyzer configured to be capable of measuring of autofluorescence spectra of cells and a cell sorter configured to be capable of sorting a desired cell population. Examples of the cell analyzer include a spectral analyzer. Furthermore, examples of the cell sorter include a cell sorter. In this embodiment, the measurement step S1 and the identification step S2 may be performed by the cell analyzer, and the sorting step S3 may be performed by the cell sorter. Alternatively, the measurement step S1 may be performed by the cell analyzer, and the identification step S2 and the sorting step S3 may be performed by the cell sorter.

### (3-1) Measurement Step

In the measurement step S1, autofluorescence spectra of the cultured photosynthetic dye-containing cell group are measured.

### (3-1-1) Culture

The culture may be culture to be performed in a culture environment in which a photosynthetic dye within a cell is induced to decrease. For example, when the photosynthetic dye-containing cell group is cultured under a nitrogen-deficient condition, the photosynthetic dye is decomposed or the production of the photosynthetic dye may be suppressed. Such a decrease in photosynthetic dye is also called whitening. In other words, the culture may be culture performed in which whitening is induced. Furthermore, the decrease in the photosynthetic dye can be induced also by culture performed under condition lacking a nutrient component (for example, phosphorus) other than nitrogen or culture performed under a condition irradiated with light of a predetermined intensity (for example, high intensity light). A decrease of a photosynthetic dye, such as whitening, is considered to occur, for example, in order to prevent excessive absorption of light energy or redistribute intracellular components (for example, intracellular nitrogen source) to sites requiring them for supporting life. As described above, the culture environment may be a culture environment in which a medium containing components controlled so as to induce a decrease of a photosynthetic dye within a cell is used, or a culture environment in which irradiation light to cells is controlled so as to induce a decrease of a photosynthetic dye within a cell.

That is, the sorting method according to the present disclosure may include a culturing step of culturing a photosynthetic dye-containing cell group under a predetermined condition. The photosynthetic dye-containing cell group cultured in the culturing step may be subjected to the measurement step.

The culture medium to be used in the culture may be appropriately selected by a person skilled in the art according to the required conditions. For example, in a case where culture is performed under a nitrogen-deficient condition, a culture medium in which a nitrogen source (for example, an ammonium salt or a nitrate salt) of a culture medium generally used for culturing a photosynthetic dye-containing cell is replaced with another component (a sodium salt or potassium salt), may be used for the culture.

As described above, the culture may be culture performed in a culture environment in which a photosynthetic dye within a cell is induced to decrease, but the culture environment may not be limited to this.

In another embodiment of the present disclosure, the culture may be culture performed in an environment that does not induce a decrease of a photosynthetic dye within a cell. In other words, the culture may be culture performed in which whitening is not induced. The culture medium for the culture may be appropriately selected by a person skilled in the art, and may be, for example, a MA2 liquid medium that will be described later. In such a culture environment, a photosynthetic dye does not decrease in most of the cells, but there are likely to have some cells having a photosynthetic-dye decrease. In addition, in such a culture environment, there are likely to appear cells having a photosynthetic-dye increase.

In the culture environment, a photosynthetic dye-containing cell may be treated with a mutagen. As the treatment with a mutagen, a treatment commonly known in the technical field may be employed. Examples of the treatment with a mutagen include, but are not limited to, UV irradiation. By the treatment with a mutagen, the probability of generating a cell having a photosynthetic-dye decrease or a cell having a photosynthetic-dye increase can be increased.

As described above, in the sorting method according to the present disclosure, culture may be performed in a culture environment in which a decrease of the photosynthetic dye within a cell is not induced. Then, cells having a photosynthetic-dye decrease or cells having a photosynthetic dye increase may be produced.

### (3-1-2) Measurement of Autofluorescence Spectra

The autofluorescence spectra of the photosynthetic dye-containing cells may be measured by the biological sample analyzer illustrated in the above (2), for example, may be measured by a cell analyzer or a cell sorter.

In order to perform the measurement, the cultured photosynthetic dye-containing cell group is supplied to flow in the channel provided in the device. Then, the light irradiation unit applies light to individual cells of the photosynthetic dye-containing cell group, and the detection unit detects light generated by the application of light. Data related to the detected light is transmitted to the information processing unit. The information processing unit generates autofluorescence spectrum data on the basis of the data related to the light.

The light to be applied by the light irradiation unit may be appropriately selected by a person skilled in the art according to the photosynthetic dye. The light irradiation unit may be configured to irradiate light that excites, for example, a phycobilin dye, a chlorophyll dye, or a carotenoid dye. More specifically, the wavelength of the excitation light may be, for example, 300 nm or greater, and preferably 400 nm or greater. Furthermore, the wavelength of the excitation light may be, for example, 800 nm or smaller, and preferably 700 nm or smaller.

Furthermore, the light detected by the light detection unit may be appropriately selected by a person skilled in the art according to the photosynthetic dye. The light detection unit may be configured to detect fluorescent light to be generated from, for example, a phycobilin dye, a chlorophyll dye, or a carotenoid dye. More specifically, the wavelength of the fluorescent light may be, for example, 500 nm or greater, and preferably 550 nm or greater. Furthermore, the wavelength of the fluorescent light may be, for example, 850 nm or smaller, and preferably 800 nm or smaller.

The information processing unit generates autofluorescence spectrum data on the basis of light data transmitted from the detection unit. A method of processing light data for acquiring the autofluorescence spectrum data may be appropriately selected by a person skilled in the art. On the basis of the light data, for example, a scattered light plot may be generated, and more specifically, a two-dimensional plot on the basis of the scattered light may be generated. A gate for a single cell is set on the scattered light plot. Then, autofluorescence spectrum data can be generated from data of an event belonging to the gate.

### (3-1-3) Examples of Samples to be Measured

In the measurement step, the autofluorescence spectra may be measured for at least two photosynthetic dye-containing cell group samples different in characteristic. The autofluorescence spectra of the at least two photosynthetic dye-containing cell group samples make the identification of the cell having a suppressed photosynthetic-dye decrease to be easier in the identification step (described later). The characteristic may be a characteristic related to a photosynthetic dye, particularly a characteristic related to the spectrum of fluorescence generated by the photosynthetic dye, and more particularly a characteristic related to the spectrum of autofluorescence.

In a preferred embodiment, the difference in characteristic may be ascribed to a difference in culture period. As described above, a decrease in photosynthetic dye may often be observed by culturing a photosynthetic dye-containing cell group under a predetermined condition. For example, a decrease in the photosynthetic dye can be observed by culture under the predetermined condition over a predetermined period. The culture period may be appropriately set by a person skilled in the art so as to obtain a desired sample. The culture period may be, for example, one week or longer, preferably two weeks or longer, three weeks or longer, or four weeks or longer. Furthermore, the upper limit value of the culture period may not be particularly set. The culture period may be, for example, 6 months or shorter, 5 months or shorter, or 4 months or shorter.

As described above, at least two photosynthetic dye-containing cell group samples different in culture period cultured under the predetermined condition are prepared. If so, it is possible to obtain at least one sample with a decrease of the photosynthetic dye (hereinafter also referred to as “photosynthetic dye-decreased sample”) and at least one sample in which the photosynthetic dye does not decrease or at least one sample with a smaller decrease of the photosynthetic dye r (hereinafter also referred to as “reference sample”). On the basis of a difference in measured autofluorescence spectrum of the photosynthetic dye-decreased sample and the reference sample, cells having a suppressed photosynthetic-dye decrease can be easily identified in the identification step (described later).

A cell population having a suppressed photosynthetic-dye decrease may be present in the photosynthetic dye-decreased sample. Therefore, the photosynthetic dye-decreased sample may be subjected to the sorting step (described later).

The reference sample may be, for example, a photosynthetic dye-containing cell group that was not subjected to culture in a culture environment in which a decrease of a photosynthetic dye is induced; a photosynthetic dye-containing cell group that was subjected to culture for a culture period shorter than that of the photosynthetic dye-decreased sample; or a cell group that was subjected to culture in a culture environment in which a decrease of a photosynthetic dye is not induced. Note that the cell type of the reference sample is preferably the same as the cell type of the photosynthetic dye-decreased sample, but may be another cell type as long as the decrease in photosynthetic dye can be identified.

In another preferred embodiment, the difference in the characteristic is ascribed to a difference in culture condition. As described above, a decrease in photosynthetic dye may often be observed by culturing a photosynthetic dye-containing cell group under a predetermined condition. On the other hand, there is a culture condition under which a photosynthetic dye does not decrease.

Then, when culture is performed under the predetermined conditions, at least one sample decreased in photosynthetic dye (“photosynthetic dye-decreased sample”) is obtained, and, when culture is performed under the condition in which a photosynthetic dye does not decrease, at least one sample having no decrease of the photosynthetic dye or having a smaller decrease of the photosynthetic dye (“reference sample”) may be obtained.

Since the culture condition differs as described above, the culture period for obtaining both samples may be the same or different. The culture period may be appropriately set by a person skilled in the art so as to obtain a desired photosynthetic dye-decreased sample. The culture period may be, for example, one week or longer, preferably two weeks or longer, three weeks or longer, or four weeks or longer. Furthermore, the upper limit value of the culture period may not be particularly set. The culture period may be, for example, 6 months or shorter, 5 months or shorter, or 4 months or shorter.

As described above, at least two photosynthetic dye-containing cell group samples different in culture condition are prepared, with the result that a photosynthetic dye-decreased sample and a reference sample can be obtained. The difference in autofluorescence spectrum measured between the photosynthetic dye-decreased sample and the reference sample makes identification of a cell having a suppressed photosynthetic-dye decrease to be easier.

As described above, examples of the sample to be measured include, but are not limited to, a photosynthetic dye-decreased sample and a reference sample.

For example, as described in the above (3-1-1), in a case where the culture is performed in the environment in which a decrease of a photosynthetic dye within a cell is not induced, the photosynthetic dye does not decrease in most of the cells, but cells in which the photosynthetic dye decreases or cells in which the photosynthetic dye increases may be produced.

As described above, at least two photosynthetic dye-containing cell group samples cultured under the culture condition different in culture period are prepared, with the result that at least one sample containing a cell having a photosynthetic-dye decrease or a cell with an increase of a photosynthetic dye (hereinafter also referred to as a “mutant-containing sample”) and at least one sample containing no cell having a photosynthetic-dye decrease or no cell with an increase of a photosynthetic dye (hereinafter also referred to as a “mutant-free sample”) can be obtained. In the measurement step, the autofluorescence spectra of the mutant-containing sample and the mutant-free sample may be measured. In the present disclosure, the mutant-containing sample and the mutant-free sample may be handled as the photosynthetic dye-decreased sample and the reference sample described above, respectively.

Furthermore, in this case, the mutant-containing sample alone may be prepared, and the autofluorescence spectra of the mutant-containing sample may be measured.

### (3-1-4) Examples of Cells

In the disclosure, the photosynthetic dye-containing cell may be an oxygenic photosynthetic cell or may be a non-oxygenic photosynthetic cell.

Examples of the cells that perform the oxygenic photosynthesis include cells of a eukaryotic photosynthetic organism and cyanobacteria. Examples of the cells of a eukaryotic photosynthetic organism include algal cells and plant cells.

Examples of the cells that perform the non-oxygenic photosynthesis include red sulfur bacteria, red non-sulfur bacteria, green sulfur bacteria, and green non-sulfur bacteria.

The photosynthetic dye-containing cell to be subjected to the sorting method according to the present disclosure may be any one of these.

In the present disclosure, the photosynthetic dye-containing cell may be, for example, an algal cell or a plant cell. In a preferred embodiment, the photosynthetic dye-containing cell is a microalgal cell. Biofuel production by a microalgal cell is consideration to be more efficient than biofuel production by a plant cell. In consideration of this, a microalgal cell is one of the particularly preferable cells to be subjected to the sorting method according to the present disclosure.

The algal cells may be, for example, prokaryotic algal cells or eukaryotic algal cells. Examples of the prokaryotic algae include cyanobacteria. The eukaryotic algae can be classified into, for example, a microalgae and a macroalgae. The microalgae and the macroalgae may be any one of red algae, brown algae, green algae, diatom, yellow-green algae, and dinoflagellate. For example, as shown in the following examples, the sorting method according to the present disclosure may be applied for sorting of red algae (e.g., Schyzon).

The plant cell may be, for example, any one of moss plant, fern plant and seed plant cells.

The photosynthetic dye may be a dye possessed by a photosynthetic organism. For example, the photosynthetic dye may contain one or more of a phycobilin dye, a chlorophyll dye, and a carotenoid dye.

Examples of the chlorophyll dye include chlorophyll and bacteriochlorophyll. The former is possessed by an oxygenic phototroph, and the latter is possessed by a non-oxygenic phototroph. Examples of the chlorophyll include chlorophyll a, chlorophyll b, chlorophyll c1, chlorophyll c2, chlorophyll d, and chlorophyll f. Examples of the bacteriochlorophyll include a bacterio-chlorophyll a, a bacterio-chlorophyll b, a bacterio-chlorophyll c, a bacterio-chlorophyll d, a bacterio-chlorophyll e, and a bacterio-chlorophyll f.

Examples of the carotenoid dye include carotene and xanthophyll. Examples of the carotene include α-carotene, β-carotene, γ-carotene, δ-carotene, and lycopene. Examples of the xanthophyll include lutein, zeaxanthin, fucoxanthin, canthaxanthin, and astaxanthin.

Examples of the phycobilin dye include phycocyanin, phycocyanobilin, phycoerythrobilin, phycoviolobilin, and phycobilin.

The photosynthetic dye-containing cell includes any one or more of these photosynthetic dyes. For example, a microalgae, Schyzon (which is a unicellular red algae) contains chlorophyll a and phycocyanin.

In the present disclosure, the cell population having a suppressed photosynthetic-dye decrease may be a cell population having a suppressed decrease of one or two or more of the photosynthetic dyes.

Note that the sorting method according to the present disclosure may be applied to cells other than the photosynthetic dye-containing cells, that is, the cells in which the autofluorescence spectra changes (decreases or increases) by culture. That is, the sorting method according to the present disclosure may be configured as a method for sorting cells with a suppressed change in autofluorescence spectrum from cultured cells.

### (2-3) Identification Step

In the identification step S2, a cell population having a suppressed photosynthetic-dye decrease (also referred to as “desired cell population” in the present specification) is identified from the photosynthetic dye-containing cell group on the basis of the autofluorescence spectra measured in the measurement step S1. The autofluorescence spectra is suitable for performing the identification.

### (3-2-1) Examples of Identifying Desired Cell Population

In the identification step, a desired cell population can be identified on the basis of difference of autofluorescence spectra of at least two photosynthetic dye-containing cell group samples different in characteristic.

For example, in the measurement step S1, an autofluorescence spectrum is measured for each of the sample with decreased photosynthetic dye and the reference sample.

When the autofluorescence spectra of the samples having a photosynthetic-dye decrease is compared with the autofluorescence spectra of the reference sample, it can be confirmed that there is a tendency that the fluorescence intensity of the autofluorescence of the samples having a photosynthetic-dye decrease is lower than that of the reference sample. This is ascribed to a decrease of a photosynthetic dye in the cells contained in the sample having a photosynthetic-dye decrease.

However, a cell population having a suppressed photosynthetic-dye decrease may be present in the photosynthetic dye-decreased sample. The cell population is a desired cell population.

The desired cell population may have autofluorescence spectra having a higher fluorescence intensity than the autofluorescence spectrum of the majority in the sample having a photosynthetic-dye decrease (or the average autofluorescence spectrum in the sample having a photosynthetic-dye decrease). The desired cell population may have autofluorescence spectra having the same fluorescence intensity as the autofluorescence spectrum of the majority in the reference sample (or the average autofluorescence spectrum of the reference sample).

Then, a cell population having autofluorescence spectra having the same fluorescence intensity as the autofluorescence spectrum of the majority in the reference sample (or the average autofluorescence spectrum of the reference sample) is identified as the desired cell population.

In the present specification, the autofluorescence spectrum of the majority may be autofluorescence spectra that are mostly present among autofluorescence spectra of all cells to be measured and contained in a sample to be measured (for example, a sample having a photosynthetic-dye decrease or a reference sample). For example, the autofluorescence spectrum of the majority may be the spectra drawn by plotting the fluorescence intensities of, for example, 10% or more, 15% or more 20% or more, 25% or more, 30% or more, 35% or more, 40% or more, 45% or more, or 50% or more cells relative to all the cells to be measured, against the wavelength.

In the present specification, the average autofluorescence spectrum may be an autofluorescence spectrum corresponding to an average of the autofluorescence spectra of all cells to be measured and contained in a sample to be measured (for example, a sample having a photosynthetic-dye decrease or a reference sample). The average autofluorescence spectrum may be, for example, a spectrum drawn by plotting an average value of fluorescence intensities measured at a certain detection wavelength (for example, an average value of fluorescence intensities of all cells) against the wavelength.

As described in the above (3-1-3), in the measurement step S1, an autofluorescence spectrum may be measured for each of the mutant-containing sample and the mutant-free sample. In this case, when the autofluorescence spectrum of the mutant-containing sample is compared with that of the mutant-free sample, it can be confirmed that the fluorescence intensity of the autofluorescence of the mutant-containing sample is lower or higher than that of the mutant-free sample. This may be because a cell population having a photosynthetic-dye decrease or a cell population with an increased photosynthetic dye is contained in the mutant-containing sample. The cell population may be identified as a desired cell population. For example, as described above, the autofluorescence spectrum of the majority of the mutant-containing sample and/or the mutant-free sample may be referred to, and a cell population having autofluorescence spectra with a lower or higher fluorescence intensity than the autofluorescence spectrum of the majority may be identified as the desired cell population. In addition, an average autofluorescence spectrum may be referred to in place of the autofluorescence spectrum of the majority.

Furthermore, as described in the above (3-1-3), in the measurement step S1, the autofluorescence spectra of the mutant-containing sample alone may be measured. In this case, only the autofluorescence spectra of the mutant-containing sample alone may be referred to. For example, a cell population having autofluorescence spectra with a fluorescence intensity lower or higher than the fluorescence intensity of the autofluorescence spectrum of the majority of the mutant-containing sample may be identified as the desired cell population.

### (3-2-2) Examples of Identification of Fluorescence Wavelength Band for Identifying Desired Cell Population

In the identification step, one or more fluorescence wavelength bands for identifying the desired cell population may be specified, and particularly, two or more (for example, two) fluorescence wavelength bands may be specified. In the present specification, the fluorescence wavelength band to be specified is also referred to as a “wavelength band for sorting”.

On the basis of the one or more wavelength bands for sorting, plot data for setting the gate to be employed in the sorting step (described later) can be generated. For example, in a case where two wavelength bands for sorting are specified, a two-dimensional plot by plotting the two wavelength bands for sorting on the vertical axis and the horizontal axis, respectively, may be formed, and a gate for identifying the desired cell population for the two-dimensional plot may be set.

For example, the one or more wavelength bands for sorting may be specified such that a desired cell population and other cell populations in the sample having a photosynthetic-dye decrease can be distinguished.

The one or more wavelength bands for sorting may be set in accordance with, for example, the fluorescence wavelength of a photosynthetic dye contained in a photosynthetic dye-containing cell, and may be specified so as to include, for example, at least a part of the fluorescence wavelength.

In addition, the one or more wavelength bands for sorting may be specified as described below but the specification method is not limited to these examples.

For example, one or more (particularly, two or more) fluorescence wavelength bands having the largest difference in fluorescence intensity between the autofluorescence spectrum of the majority in the samples having a photosynthetic-dye decrease and the autofluorescence spectra of the desired cell population may be specified as the one or more wavelength bands for sorting.

Alternatively, one or more (particularly, two or more) fluorescence wavelength bands having the largest difference in fluorescence intensity between the average autofluorescence spectrum and the autofluorescence spectrum of the desired cell population may be specified as the one or more wavelength bands for sorting.

As described above, the one or more wavelength bands for sorting may be specified on the basis of the difference in fluorescence intensity in the autofluorescence spectra.

Furthermore, for example, one or more (particularly, two or more) fluorescence wavelength bands having the largest difference in fluorescence intensity between the autofluorescence spectrum of the majority in the sample having a photosynthetic-dye decrease and the autofluorescence spectrum of the majority in the reference sample may be specified as the one or more wavelength bands for sorting.

Alternatively, one or more (particularly, two or more) fluorescence wavelength bands having the largest difference in fluorescence intensity between the average autofluorescence spectrum in the sample having a photosynthetic-dye decrease and the average autofluorescence spectrum of the reference sample may be specified as the one or more wavelength bands for sorting.

The reference sample has an autofluorescence spectrum similar to that of the desired cell population. Therefore, as described above, the one or more wavelength bands for sorting may be specified on the basis of the difference in fluorescence intensity between the autofluorescence spectra of the sample having a photosynthetic-dye decrease and that of the reference sample.

The desired cell population may be identified on the basis of the light data (fluorescence intensity) in one or more wavelength bands for sorting specified as described above.

As described in the above (3-1-3), in the measurement step S1, an autofluorescence spectrum may be measured for each of the mutant-containing sample and the mutant-free sample. Also in this case, the wavelength band for sorting may be specified such that the desired cell population and other cell populations in the mutant-containing sample can be distinguished, as described above. For example, one or more (particularly, two or more) fluorescence wavelength bands having the largest difference in fluorescence intensity between the autofluorescence spectrum of the majority in the mutant-containing sample and the autofluorescence spectrum of the desired cell population may be specified as the one or more wavelength bands for sorting. Alternatively, one or more (particularly, two or more) fluorescence wavelength bands having the largest difference in fluorescence intensity between the average autofluorescence spectrum and the autofluorescence spectrum of the desired cell population may be specified as the one or more wavelength bands for sorting.

Furthermore, as described in the above (3-1-3), in the measurement step S1, the autofluorescence spectra of the mutant-containing sample alone may be measured. Also in this case, the wavelength band for sorting may be specified such that the desired cell population and other cell populations in the mutant-containing sample can be distinguished, as described above.

### (3-2-3) Examples of Gate Setting for Identifying Desired Cell Population

In the present technology, the desired cell population (that is, a cell population having a suppressed photosynthetic-dye decrease may be identified by gate setting for data of the measured autofluorescence spectra. The gate setting may be performed on, for example, data related to autofluorescence spectra, for example, on plot data (for example, two-dimensional plot data) or spectrum data. A sorting target in the sorting step (described later) is identified by the gate setting.

More specifically, a gate for identifying a desired cell population is set for the data of the autofluorescence spectrum of the sample having a photosynthetic-dye decrease described above.

The gate for identifying the desired cell population may be a gate covering, for example, the autofluorescence spectrum of the majority of the autofluorescence spectra of the reference sample (or the average autofluorescence spectrum of the reference sample).

Furthermore, the gate for identifying the desired cell population may be a gate excluding, for example, the autofluorescence spectrum of the majority from autofluorescence spectra of the samples having a photosynthetic-dye decrease (or the average autofluorescence spectrum in the sample having a photosynthetic-dye decrease).

In one embodiment, for the gate setting, in the one or more wavelength bands for sorting specified as described above, a cell population having a same-level fluorescence intensity as that of the reference sample and a cell population having a same-level fluorescence intensity as that of the majority of the sample having a photosynthetic-dye decrease can be specified. The cell population having a same-level of fluorescence intensity as that of the reference sample may be identified as a cell population having a suppressed photosynthetic-dye decrease and a gate may be set for the cell population.

As described in the above (3-1-3), in the measurement step S1, an autofluorescence spectrum may be measured for each of the mutant-containing sample and the mutant-free sample. Also in this case, the gate setting is performed as mentioned above; that is, a gate for identifying a desired cell population is set for the data of the autofluorescence spectrum of a mutant-containing sample. For example, the gate may be set so as not to cover the autofluorescence spectrum of the majority in the mutant-containing sample. Moreover, the gate may be set so as not to cover the autofluorescence spectrum of the majority in the mutant-free sample.

Furthermore, as described in the above (3-1-3), in the measurement step S1, the autofluorescence spectra of the mutant-containing sample alone may be measured. Also in this case, the wavelength band for sorting may be specified such that the desired cell population and other cell populations in the mutant-containing sample can be distinguished, as described above. For example, the gate may be set so as not to cover the autofluorescence spectrum of the majority in the mutant-containing sample.

### (3-3) Sorting Step

In the sorting step S3, the cell population having a suppressed photosynthetic-dye decrease (that is, a desired cell population) identified in the identification step S2 is sorted. For example, in the sorting step S3, cells belonging to the gate set in the identification step S2 are sorted from the sample having a photosynthetic-dye decrease. The sorted cell population is the desired cell population.

The sorting of the cells belonging to the gate may be appropriately performed by a person skilled in the art using the biological sample analyzer illustrated in the above (2).

For example, in order to perform the sorting, the sample having a photosynthetic-dye decrease is supplied to flow in the channel provided in the device. Then, the light irradiation unit applies light to individual cells of the sample having a photosynthetic-dye decrease and the detection unit detects light generated by the application of light. Detected light is transmitted as light data to the information processing unit. The information processing unit determines whether or not each cell is a cell belonging to the gate on the basis of the light data. Then, in a case where it is determined that a cell belongs to the gate, the information processing unit makes the sorting unit to perform processing of sorting the cell.

In the method according to the present disclosure, a confirmation step of confirming whether or not the cell sorted in the sorting step S3 belongs to a cell population having a suppressed photosynthetic-dye decrease, that is, whether or not the cell is a desired cell, may be performed. In the confirmation step, the autofluorescence spectra of the sorted cell population are measured as described in the measurement step S1. Whether or not the sorted cell population is a desired cell population is confirmed on the basis of the measured autofluorescence spectra.

### (4) Examples

Photosynthetic dye-containing cells such as microalgal cells are expected to be utilized in biofuel extraction. In addition, utilization of the dye itself contained in photosynthetic dye-containing cells is also expected. In order to utilize the cells in this way, it is desirable that the cells can be stably cultured.

However, in a case where the photosynthetic dye-containing cell is cultured, the photosynthetic dye may sometimes be decomposed. For example, microalgal cells are often cultured in a nitrogen-deficient environment for biofuel extraction but a photosynthetic dye sometimes decreases by, e.g., decomposition.

Therefore, it is considered extremely useful if a photosynthetic dye-containing cell having a suppressed photosynthetic-dye decrease can be obtained.

One of the promising microalgae as a suitable organism for biofuel production is a unicellular red algae Cyanidioschyzon merolae (also referred to herein as Schyzon). Schyzon shows various nitrogen deficiency responses when nitrogen lacks in the environment. One of the nitrogen deficiency responses is decomposition of a photosynthetic dye. That is, Schyzon decomposes a photosynthetic dye in the absence of nitrogen. As a result, the color of the cell changes from green to yellowish green in several days, and then, into white over several weeks.

Schyzon having a suppressed photosynthetic-dye decrease in the absence of nitrogen is considered useful for biofuel production. Then, the sorting method according to the present disclosure was performed using Schyzon.

Schyzon was cultured in a cotton-filter tube for 0, 1, 2, or 4 weeks under a nitrogen-deficient culture condition. The details of the cultured Schyzon and the culture condition thereof are as follows.

Schyzon: a wild-type strain of Cyanidioschyzon merolae 10D (Kuroiwa, 1998) (also referred to as a WT strain)

Culture medium: Nitrogen-deficient liquid medium (MA2 liquid medium was modified so as not to contain a nitrogen source. In order to obtain a nitrogen-deficient culture condition, neither an ammonium salt nor a nitrate serving as a nitrogen source is contained in the MA2 liquid medium. The method for producing the medium is as described below.)

Culture environment: culture in a cotton-filter tube (40° C., 2% CO₂ aeration, 50 μmol photons·m⁻²·s⁻¹ white-light condition)

The nitrogen-deficient liquid medium was produced as follows.

That is, the following −N Solution I, MA2 Solution II, MA2 Solution III and H₂O were mixed, and the resulting mixture was autoclaved. After the autoclaving, the following MA2 Solution IV sterilized with a filter was added to the mixture to obtain a nitrogen-deficient liquid medium. The compositions of individual Solutions are as follows.

TABLE 1
Nitrogen-deficient liquid medium

| Name of Reagent | Volume |
|---|---|
| 10 × -N Solution I | 100 mL |
| 100 × MA2 Solution II | 10 mL |
| 1000 × MA2 Solution III | 1 mL |
| 250 × MA2 Solution IV | 4 mL |
| H₂O | 885 mL |

TABLE 2
500× A6 minor salts

| | | Final |
|---|---|---|
| Name of Reagent | Volume | Concentration |
| H₂BO₃ | 2.85 g | 46 mM |
| MnCl₂•4H₂O | 1.8 g | 9 mM |
| ZnCl₂ | 0.105 g | 0.77 mM |
| Na₂MoO₄•2H₂O | 0.39 g | 1.6 mM |
| CoCl₂•6H₂O | 0.04 g | 0.17 mM |
| CuCl₂ | 0.043 g | 0.3 mM |

TABLE 3
10× -N Solution I

| | | Final |
|---|---|---|
| Name of Reagent | Volume | Concentration |
| Na₂SO₄ | 56.8 g | 40 mM |
| MgSO₄•7H₂O | 10.0 g | 4 mM |
| A6 minor salts | 40 mL | |
| H₂SO₄ | 3 mL | |
| Water up to 1 L | | |

TABLE 4
100× MA2 Solution II

| | | Final |
|---|---|---|
| Name of Reagent | Volume | Concentration |
| KH₂PO₄ | 10.88 g | 800 mM |
| Water up to 100 ml | | |

TABLE 5
1000× MA2 Solution III

| | | Final |
|---|---|---|
| Name of Reagent | Volume | Concentration |
| CaCl₂•2H₂O | 14.7 g | 1M |
| Water up to 100 ml | | |

TABLE 6
250× MA2 Solution IV

| | | Final |
|---|---|---|
| Name of Reagent | Volume | Concentration |
| FeCl₃•6H₂O | 0.68 g | 25 mM |
| Na₂EDTA | 0.74 g | 20 mM |
| Water up to 100 mL | | |

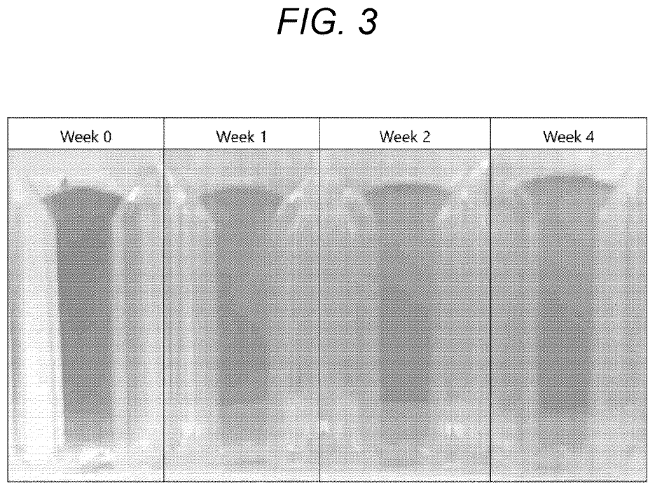

When Schyzon was cultured in a cotton-filter tube for 0, 1, 2, or 4 weeks under the nitrogen deficient culture condition, the longer the nitrogen deficient state continued, the more a color change of the culture solution proceeded from green to yellowish green was visually confirmed. In other words, a decrease (in particular, decomposition) of the photosynthetic dye was confirmed. Photographs of the culture solutions are shown in FIG. 3.

The autofluorescence spectra of cultured Schyzon (Schyzon cultured for 0, 1, 2, or 4 weeks is referred to as −N week 0, 1, 2, or 4, respectively) were measured. The measurement was performed as follows.

The culture solution was passed through a cell strainer (pluriStrainer (trademark) 5 μm, pluriSelect Life Science UG (haftungsbeschrankt) & Co. KG) in order to remove Schyzon aggregates in the culture solution. A part of the culture solution passed through the cell strainer was taken and placed on OneCell counter (OneCell Inc.), and the number of cells was counted under a microscope to determine the cell concentration in the culture solution. On the basis of the cell concentration, the culture solution was diluted to about 1×10⁷ cells/mL to obtain a diluted solution. The diluted solution was used as a sample for autofluorescence spectrum measurement. As a biological sample analyzer for measuring an autofluorescence spectrum, a spectral analyzer (Spectral Cell Analyzer SA3800, Sony Group Corporation) was used. The sample (2 mL) was dispensed into 5 mL tubes for exclusive use of for the analyzer and set in the analyzer. For the measurement of an autofluorescence spectrum, 488 nm and 638 nm excitation lasers were used. Scattered-light plots were prepared on the basis of data obtained from the measured forward scattered light (also referred to as FSC) and side scattered light (also referred to as SSC). Using the scattered light plots, data of cell masses composed of two or more cells were removed and the autofluorescence spectrum of the sample was obtained.

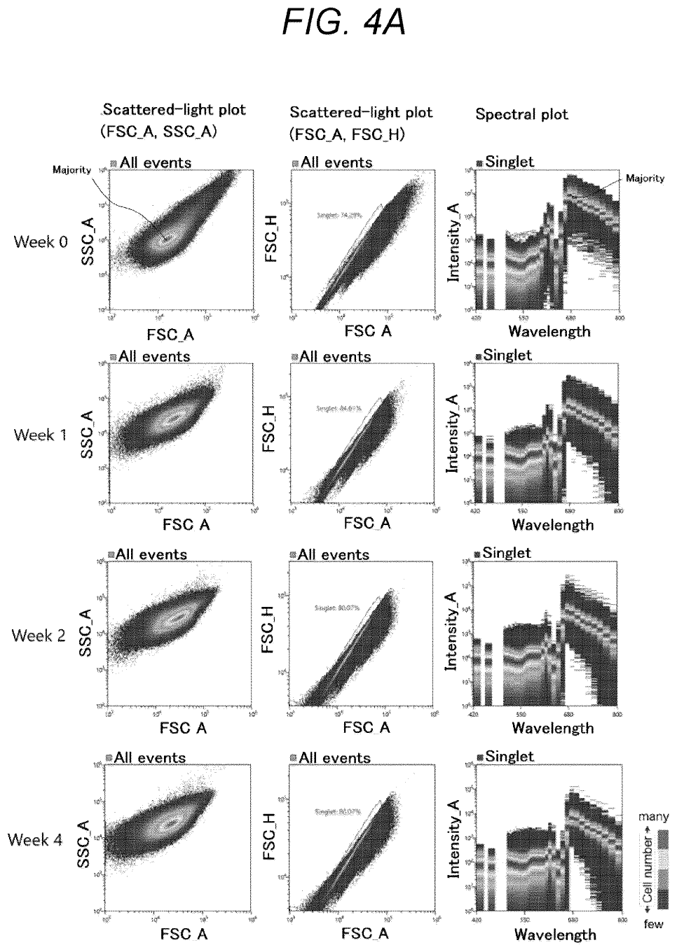

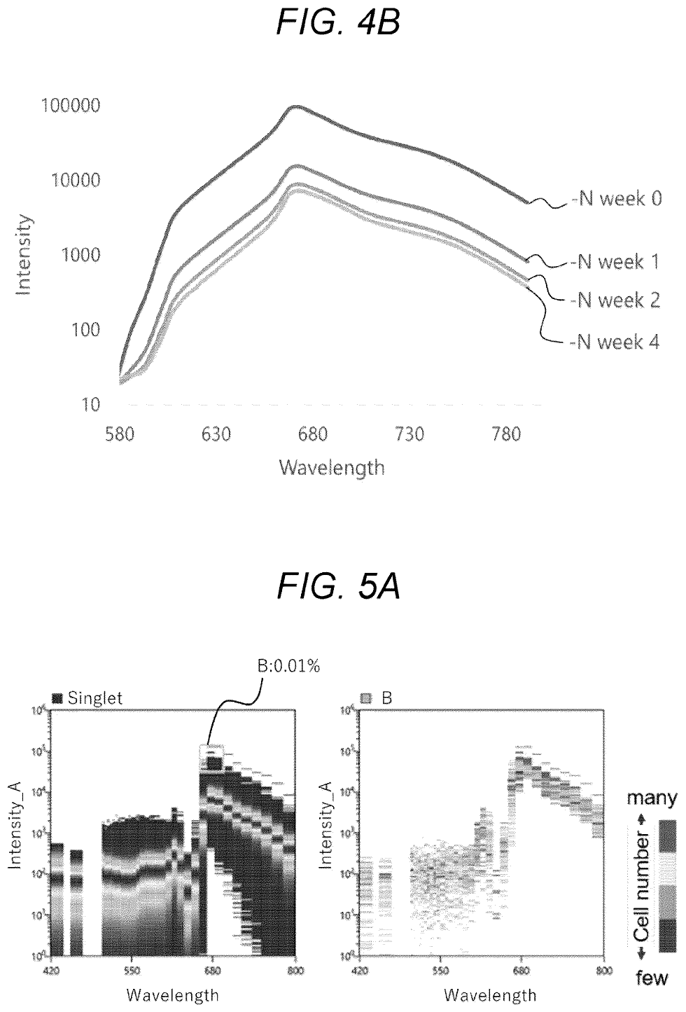

The measurement results are shown in FIGS. 4A and B.

FIG. 4A shows autofluorescence spectral plots of single cells of individual culture solutions. Measurement results of −N weeks 0, 1, 2, and 4 are shown in order from the top. The number of measured cells was 2.5 million. The left columns show scattered light plots where the horizontal axis represents FSC_A and the vertical axis represents SSC_A. The center columns show scattered light plots where the vertical axis represents FSC_H on and the horizontal axis represents FSC_A. The right column show spectral plots where the vertical axis represents fluorescence intensity and the horizontal axis represents wavelength. The spectral plots were prepared by extracting only single cells (gate indicated by Singlet in scattered light plots (FSC_A, FSC_H)) from the results of the scattered light plots (FSC_A, FSC_H).

In the scattered light plots in the left column, the central portion of the dot group spreading in a substantially elliptical shape is the spectrum of the majority. As plots away from the center toward the periphery, the number of cells having the spectrum of the value decreases. In the spectral plots in the right column, the central portion in the vertical direction is the spectrum of the majority. As plots are away from the center in the vertical direction, the number of cells having the spectrum of the value decreases. The numbers of plots are 1,857,225; 2,115,228; 2,150,198; and 2,001,770 from the top, respectively. FSC: forward scattered light, SSC: side scattered light, A: Area (area of pulse), H: Height (height of pulse).

FIG. 4B shows the spectra of the average values (that is, the average autofluorescence spectra) of the spectral plots of individual solutions. In the figure, the vertical axis represents fluorescence intensity, and the horizontal axis represents wavelength.

As shown in FIG. 4A, it was confirmed that the scattered light pattern was changed by performing culture under a nitrogen-deficient condition. Next, when autofluorescence spectra plots were prepared by taking out only single cells, the peak of fluorescence intensity (in particular, fluorescence intensity around 670 nm) became lower as the culture period under the nitrogen deficient condition increased, as shown in FIG. 4A and FIG. 4B. For example, the fluorescence intensity of the majority at a wavelength of around 670 nm in the case of −N week 0, is about 10⁵, whereas the fluorescence intensity of the majority at a wavelength of around 670 nm in the case of −N week 4 is about 10⁴.

Schyzon contains chlorophyll a (also referred to as Ch1 a in the present specification) and phycocyanin (also referred to as PC) as photosynthetic dyes. The fluorescence wavelengths of Ch1 a and PC are from 635 to 650 nm and from 665 to 675 nm, respectively. Therefore, it is considered that the fluorescence intensity decreased because these photosynthetic dyes decreased (particularly decomposed) by the culture under the nitrogen-deficient condition.

Identification of cells to be sorted and sorting of the identified cells were performed using −N week 4 having the lowest peak as a sample having a decreased photosynthetic dye, and −N week 0 (a culture period of 0 week) as a reference sample. The identification and sorting were performed as follows.

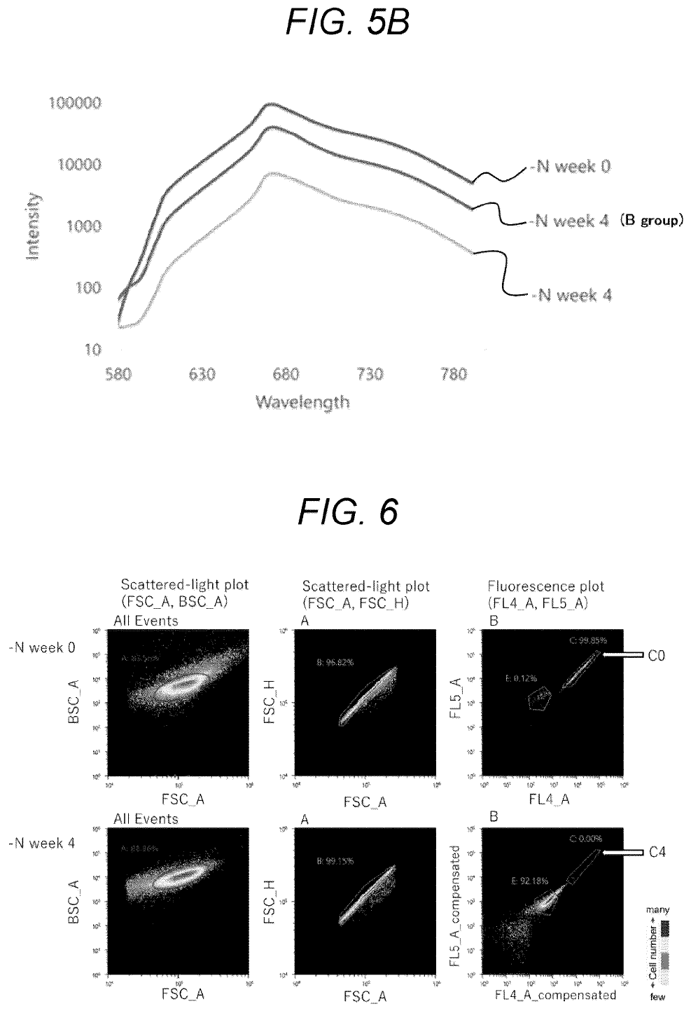

FIG. 5A, left shows autofluorescence spectral plots at −N week 4. A cell having the same spectrum as that of −N week 0 among these spectral plots was identified as a cell having a suppressed photosynthetic-dye decrease (particularly, a cell with suppressed decomposition of photosynthetic dyes). The identified cells are identified in the figure by gate B. In addition, a spectral plot obtained by extracting only gate B is illustrated on the right side of the figure. Also, FIG. 5B shows the average autofluorescence spectra of the cell populations −N week 0, −N week 4, and −N week 4 (B group)) identified by gate B. Among the 2.5 million cells at −N week 4, the number of cells identified by gate B was 115 cells, that is, the proportion of cells having a suppressed photosynthetic-dye decrease was 0.0046%.

Next, cells having a suppressed photosynthetic-dye decrease were sorted. For the sorting, a cell sorter (Cell Sorter SH800S, Sony Group Inc.) was used as a biological sample analyzer. For sorting by the cell sorter, an excitation laser similar to that used for the autofluorescence spectra measurement described above was used. In addition, two bandpass filters of 665/30BP and 720/60BP were used to distinguish the cells of the experimental purpose. The cell sorting conditions were 70 μm sorting chip, sort mode Normal, Sample Pressure 4, and room temperature.

The measurement results of autofluorescence spectra of −N week 0 and −N week 4 by a cell sorter are shown in FIG. 6. In the measurement, 720±30 nm (FL5 detector was assigned) and 665±15 nm (FL4 detector) were used as wavelength bands for sorting. The number of measured cells was 100,000.

The upper stage of the figure shows autofluorescence spectra of −N week 0 (reference sample).

The upper left column shows a scattered light plot of all the cells to be measured, where the vertical axis represents BSC_A and the horizontal axis represents FSC_A.

The upper center column shows living cell scattered light plots where the vertical axis represents FSC_H and the horizontal axis represents FSC_A, and was prepared by developing an elliptical gate A (gate for living cells) set in the scattered light plot of all cells in the upper left column. The number of plots in the upper center column was 83,556.

The upper right column shows plots related to the fluorescence intensities of the two wavelength bands for sorting, where the vertical axis represents FL5_A and the horizontal axis represents FL4_A. The plots were prepared by developing gate B set for single cells alone in the living cell scattered light plot. The number of plots was 80, 903. Gate C (indicated by arrow CO) in the plots is set for the majority cell population of the reference sample. Gate E is a gate set for a cell population having low fluorescence intensity. The numbers of plots in gate C and gate E were 80,782 and 98, respectively.

The meanings of the abbreviations in the figure are as follows. BSC: backward scattered light, FSC: forward scattered light, SSC: lateral scattered light, A: Area (area of pulse), H: Height (height of pulse).

The lower stage of the figure shows autofluorescence spectra of −N week 4 (sample having a photosynthetic-dye decrease). The lower left column shows scattered light plots of all the cells to be measured where the vertical axis represents BSC_A and the horizontal axis represents FSC_A.

The lower center column shows living cell scattered light plots where the vertical axis represents FSC_H and the horizontal axis represents FSC_A, and was prepared by developing an elliptical gate A (gate for living cells) set in the scattered light plots of all cells in the lower left column. The number of plots in the lower center column was 88,856.

The lower right column shows plots related to the fluorescence intensities of the two wavelength bands for sorting, where the vertical axis represents FL5_A and the horizontal axis represents FL4_A. The plots were prepared by developing gate B set for single cells alone in the living cell scattered light plot. The number of plots was 88,105. Gate C (indicated by arrow C4) in the plots is set for the cell population having a suppressed photosynthetic-dye decrease. Gate E is a gate set for a cell population having low fluorescence intensity. The numbers of plots in gate C and gate E were 4 and 81, 217, respectively.

Of −N week 4 (sample having a photosynthetic-dye decrease), cells belonging to gate C were sorted by the cell sorter as cells having a suppressed photosynthetic-dye decrease. The sorted cells were collected in MA2 liquid medium (100 μL) dispensed to a 96-well plate. Also, a part of the sorted cells was collected in the MA2 liquid medium (1 mL) dispensed to a 5 mL-tube for exclusive use of for the spectral analyzer. The MA2 liquid medium was prepared in the same manner as in the nitrogen-deficient liquid medium except that the following MA2 Solution I medium was used in place of −N Solution I used for preparing the nitrogen-deficient liquid medium described above.

TABLE 7
10× MA2 Solution I

| | | Final |
|---|---|---|
| Name of Reagent | Volume | Concentration |
| (NH₄)₂SO₄ | 52.9 g | 40 mM |
| MgSO₄•7H₂O | 10.0 g | 4 mM |
| A6 minor salts | 40 mL | |
| H₂SO₄ | 3 mL | |
| Water up to 1 L | | |

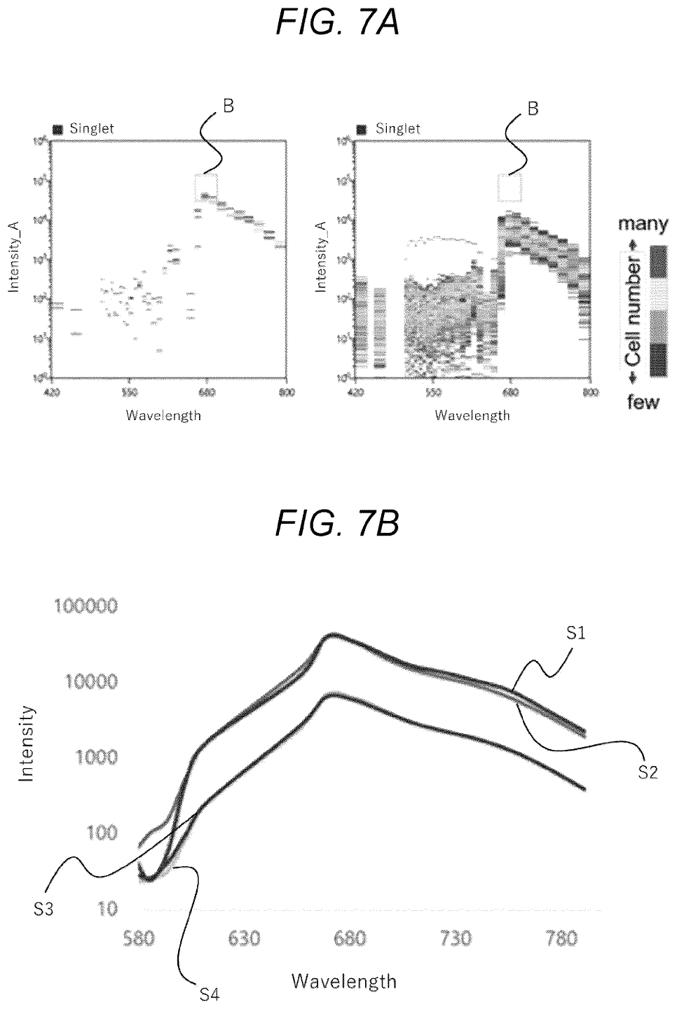

The autofluorescence spectra of the cells collected in the 5-mL tube were measured again by the spectral analyzer (Spectral Cell Analyzer SA3800, Sony Group Corporation). The measurement results are shown in FIGS. 7A and B.

FIG. 7A, left shows the autofluorescence spectra of the cell population sorted by gate C, that is, the cell population having a suppressed photosynthetic-dye decrease. The number of measured cells is 12, and the number of plots is 4. Gate B on the graph indicates the position of the spectrum of the cell population having a suppressed photosynthetic-dye decrease.

FIG. 7A, right shows the autofluorescence spectra of the cell population sorted by gate E, that is, the cell population having a photosynthetic-dye decrease. The number of measured cells is 722, and the number of plots is 703. The gate B on the graph is similar to that on the left graph (Singlet), but no plot was present in gate B.

FIG. 7B shows the measurement results of an averaged autofluorescence spectrum. S1 shows the average autofluorescence spectrum of the cell population sorted by gate C, that is, the cell population having a suppressed photosynthetic-dye decrease. Furthermore, S3 shows the autofluorescence spectrum of the cell population sorted by gate E, that is, the cell population having a photosynthetic-dye decrease.

In FIG. 7B, S2 shows the average autofluorescence spectrum of −N week 4 (B group) shown in FIG. 5B. Furthermore, S4 shows an average autofluorescence spectrum of −N week 4.

The average autofluorescence spectrum shown by S1 is almost equivalent to that shown by S2. Furthermore, the average autofluorescence spectrum shown by S3 is almost equivalent to that shown by S4. From these results, it was confirmed that the population of cells having a suppressed photosynthetic-dye decrease was sorted.

As described above, it is possible to sort a Schyzon cell population having a suppressed photosynthetic-dye decrease by the sorting method according to the present disclosure. Schyzon has been used as a model organism for eukaryotic photosynthetic organisms. Therefore, it is considered that the sorting method according to the present disclosure can be applied to various photosynthetic dye-containing cells.

### 2. Cell Sorter

The present disclosure also provides a cell sorter configured to perform a sorting step of sorting a cell population having a suppressed photosynthetic-dye decrease from a photosynthetic dye-containing cell group. Herein, the cell population having a suppressed photosynthetic-dye decrease may be a cell population identified on the basis of the autofluorescence spectra of the cultured photosynthetic dye-containing cell group.

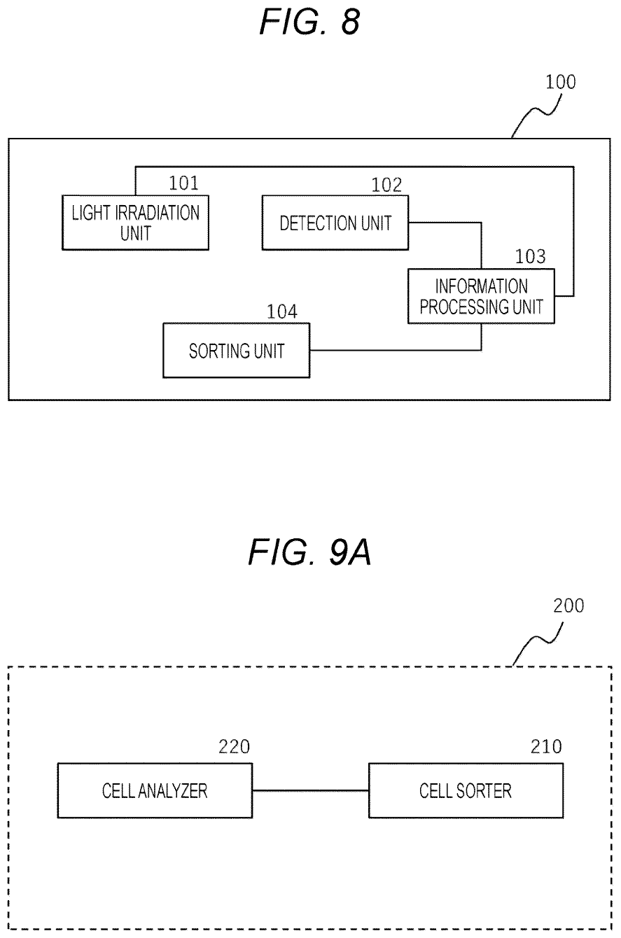

The cell sorter may be configured as the biological sample analyzer described above. A configuration of the cell sorter is illustrated in FIG. 8. As illustrated in the figure, the cell sorter 100 may include a light irradiation unit 101, a detection unit 102, an information processing unit 103, and a sorting unit 104. The light irradiation unit 101, the detection unit 102, the information processing unit 103, and the sorting unit 104 may be the same as the light irradiation unit 6101, the detection unit 6102, the information processing unit 6103, and the sorting unit 6104 described in the above Section 1 and the descriptions regarding these units also apply to the cell sorter according to the present disclosure. The cell sorter may be, for example, a so-called spectral cell sorter.

The cell sorter may be configured to perform the sorting method according to the present disclosure, and may be configured to perform the sorting step S103 described in the above Section 1. The cell sorter may be configured to perform the measurement step S101 and/or the identification step S102 in addition to the sorting step.

### 3. Cell Sorting System

The present disclosure also provides a cell sorting system including a cell sorter configured to perform a sorting step of sorting a cell population having a suppressed photosynthetic-dye decrease from a photosynthetic dye-containing cell group. Herein, the cell population having a suppressed photosynthetic-dye decrease may be a cell population identified on the basis of the autofluorescence spectra of the cultured photosynthetic dye-containing cell group.

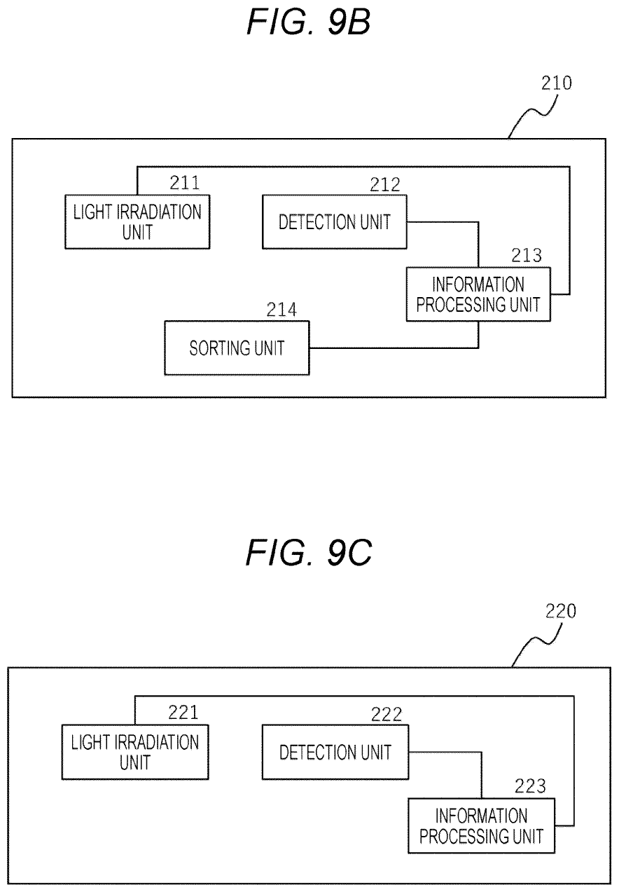

A configuration of the cell sorting system is illustrated in FIG. 9A. As shown in the figure, the cell sorting system 200 includes a cell sorter 210 and a cell analyzer 220.

A configuration of the cell sorter 210 is illustrated in FIG. 9B. As illustrated in the figure, the cell sorter 210 may include a light irradiation unit 211, a detection unit 212, an information processing unit 213, and a sorting unit 214. The light irradiation unit 211, the detection unit 212, the information processing unit 213, and the sorting unit 214 may be the same as the light irradiation unit 6101, the detection unit 6102, the information processing unit 6103, and the sorting unit 6104 described in the above Section 1 and the descriptions regarding these units also apply to the cell sorter. The cell sorter 210 may be, for example, a cell sorter.

A configuration of the cell analyzer 220 is illustrated in FIG. 9C. As illustrated in the figure, the cell analyzer 220 may include a light irradiation unit 221, a detection unit 222 and an information processing unit 223. The light irradiation unit 221, the detection unit 222 and the information processing unit 223, may be the same as the light irradiation unit 6101, the detection unit 6102 and the information processing unit 6103, described in the above Section 1 and the descriptions regarding these units also apply to the cell sorter. The cell analyzer 220 may be, for example, a so-called spectral analyzer.

The cell sorter 210 may be configured to perform the sorting step S103 described in the above Section 1. The cell analyzer 220 may be configured to perform the measurement step S101 and/or the sorting step S102 described in the above Section 1. As described above, the cell sorting system according to the present disclosure may be configured to perform the sorting method according to the present disclosure by a combination of the cell sorter and the cell analyzer.

Note that the present disclosure can also have the following constitutions.
[1]

A method for sorting a photosynthetic dye-containing cell, including
a measurement step of measuring autofluorescence spectra of a cultured photosynthetic dye-containing cell group; and
a sorting step of sorting a cell population having a suppressed photosynthetic-dye decrease from the photosynthetic dye-containing cell group on the basis of the measured autofluorescence spectra.
[2]

The sorting method according to [1], in which in the measurement step, autofluorescence spectra of at least two photosynthetic dye-containing cell group samples different in characteristic are measured.
[3]

The sorting method according to [2], in which the difference in characteristic is ascribed to a difference in culture period.
[4]

The sorting method according to [2] or [3], in which the cell population having a suppressed photosynthetic-dye decrease is a cell population identified on the basis of a difference in autofluorescence spectra of the at least two photosynthetic dye-containing cell group samples.
[5]

The sorting method according to any one of [1] to [4], in which the cell population having a suppressed photosynthetic-dye decrease is identified by gate setting for data of the measured autofluorescence spectra.
[6]

The sorting method according to any one of [1] to [5], in which the culture is culture performed in a culture environment in which a photosynthetic dye within a cell is induced to decrease.
[7]

The sorting method according to [6], in which the culture environment is a culture environment using a medium that contains components adjusted so as to induce a decrease of a photosynthetic dye within a cell, or a culture environment using cell-irradiation light adjusted so as to induce a decrease of a photosynthetic dye within a cell.
[8]

The sorting method according to any one of [1] to [7], further including
an identification step of identifying a cell population having a suppressed photosynthetic-dye decrease on the basis of the measured autofluorescence spectra, in which
the cell population identified in the identification step is sorted in the sorting step.
[9]

The sorting method according to [8], in which
the identification step is performed by a cell analyzer, and the sorting step is performed by a cell sorter, or
both the identification step and the sorting step are performed by a cell sorter.
[10]

The sorting method according to any one of [1] to [9], in which the photosynthetic dye-containing cell is an algal cell or a plant cell.
[11]

The sorting method according to any one of [1] to [9], in which the photosynthetic dye-containing cell is a microalgal cell.
[12]

The sorting method according to any one of [1] to [11], in which the photosynthetic dye-containing cell is an oxygenic photosynthetic cell or a non-oxygenic photosynthetic cell.
[13]

The sorting method according to any one of [1] to [12], in which the photosynthetic dye is a dye possessed by a photosynthetic organism.
[14]

The sorting method according to any one of [1] to [13], in which the photosynthetic dye includes one or more of a phycobilin dye, a chlorophyll dye, and a carotenoid dye.
[15]

A method for sorting a photosynthetic dye-containing cell, including
a sorting step of sorting a cell population having a suppressed photosynthetic-dye decrease from the photosynthetic dye-containing cell group, in which
the cell population having a suppressed photosynthetic-dye decrease is a cell population identified on the basis of autofluorescence spectra of the cultured photosynthetic dye-containing cell group.
[16]

A cell sorter
configured to perform a sorting step for sorting
a cell population having a suppressed photosynthetic-dye decrease from the photosynthetic dye-containing cell group, in which the cell population having a suppressed photosynthetic-dye decrease is a cell population identified on the basis of the autofluorescence spectra of the cultured photosynthetic dye-containing cell group.
[17]

A cell sorting system including
a cell sorter configured to perform a sorting step of soring a cell population having a suppressed photosynthetic-dye decrease from the photosynthetic dye-containing cell group, in which
the cell population having a suppressed photosynthetic-dye decrease is a cell population identified on the basis of the autofluorescence spectra of the cultured photosynthetic dye-containing cell group.

### REFERENCE SIGNS LIST

100 Cell sorter
101 Light Irradiation Unit
102 Detection Unit
103 Information Processing Unit
104 Sorting Unit
200 Sample Sorting System
210 Cell sorter
220 Cell analyzer

1. A method for sorting a photosynthetic dye-containing cell, comprising
a measurement step of measuring autofluorescence spectra of a cultured photosynthetic dye-containing cell group; and
a sorting step of sorting a cell population having a suppressed photosynthetic-dye decrease from the photosynthetic dye-containing cell group on a basis of the measured autofluorescence spectra.

2. The sorting method according to claim 1, wherein, in the measurement step, autofluorescence spectra of at least two photosynthetic dye-containing cell group samples different in characteristic are measured.

3. The sorting method according to claim 2, wherein the difference in characteristic is ascribed to a difference in culture period.

4. The sorting method according to claim 2, wherein the cell population having a suppressed photosynthetic-dye decrease is a cell population identified on a basis of a difference in autofluorescence spectra of the at least two photosynthetic dye-containing cell group samples.

5. The sorting method according to claim 1, wherein the cell population having a suppressed photosynthetic-dye decrease is identified by gate setting for data of the measured autofluorescence spectra.

6. The sorting method according to claim 1, wherein the culture is culture performed in a culture environment in which a photosynthetic dye within a cell is induced to decrease.

7. The sorting method according to claim 6, wherein the culture environment is a culture environment using a medium that contains components adjusted so as to induce a decrease of a photosynthetic dye within a cell, or a culture environment using cell-irradiation light adjusted so as to induce a decrease of a photosynthetic dye within a cell.

8. The sorting method according to claim 1, further comprising
an identification step of identifying a cell population having a suppressed photosynthetic-dye decrease on a basis of the measured autofluorescence spectra, wherein
the cell population identified in the identification step is sorted in the sorting step.

9. The sorting method according to claim 8, wherein
the identification step is performed by a cell analyzer, and the sorting step is performed by a cell sorter, or
both the identification step and the sorting step are performed by a cell sorter.

10. The sorting method according to claim 1, wherein the photosynthetic dye-containing cell is an algal cell or a plant cell.

11. The sorting method according to claim 1, wherein the photosynthetic dye-containing cell is a microalgal cell.

12. The sorting method according to claim 1, wherein the photosynthetic dye-containing cell is an oxygenic photosynthetic cell or a non-oxygenic photosynthetic cell.

13. The sorting method according to claim 1, wherein the photosynthetic dye is a dye possessed by a photosynthetic organism.

14. The sorting method according to claim 1, wherein the photosynthetic dye comprises one or more of a phycobilin dye, a chlorophyll dye, and a carotenoid dye.

15. A method for sorting a photosynthetic dye-containing cell, comprising
a sorting step of sorting a cell population having a suppressed photosynthetic-dye decrease from the photosynthetic dye-containing cell group, wherein
the cell population having a suppressed photosynthetic-dye decrease is a cell population identified on a basis of the autofluorescence spectra of the cultured photosynthetic dye-containing cell group.

16. A cell sorter
configured to perform a sorting step of sorting a cell population having a suppressed photosynthetic-dye decrease from the photosynthetic dye-containing cell group, wherein
the cell population having a suppressed photosynthetic-dye decrease is a cell population identified on a basis of the autofluorescence spectra of the cultured photosynthetic dye-containing cell group.

17. A cell sorting system comprising
a cell sorter configured to perform a sorting step of sorting a cell population having a suppressed photosynthetic-dye decrease from the photosynthetic dye-containing cell group, wherein
the cell population having a suppressed photosynthetic-dye decrease is a cell population identified on a basis of the autofluorescence spectra of the cultured photosynthetic dye-containing cell group.
